(19) United States
(12) Patent Application Publication
(10) Pub. No.: US 2026/0294303 A1
(43) Pub. Date: Oct. 1, 2026

(54) APPARATUS, METHOD, AND NON-TRANSITORY COMPUTER-READABLE MEDIUM FOR PERFORMING LEARNING PROCESSING OF MACHINE LEARNING MODEL

(71) Applicant: Yokogawa Electric Corporation, Tokyo (JP)

(72) Inventors: Kiyomi MURAOKA, Tokyo (JP); Hideo UEMURA, Tokyo (JP); Kie SHIMAMURA, Tokyo (JP)

(21) Appl. No.: 19/575,862

(22) Filed: Mar. 23, 2026

(30) Foreign Application Priority Data
Mar. 25, 2025 (JP) 2025-049400

(51) Int. Cl. A61B 5/16; G06N 20/00

(52) U.S. Cl. CPC A61B 5/165; G06N 20/00

(57) ABSTRACT

Provided is an apparatus comprising a processor, wherein the processor receives measured data of brain waves of a subject, estimates an estimated state of the subject according to the measured data of brain waves of the subject by using a machine learning model which calculates, upon input of measured data of brain waves, an estimated state of at least one of a physical state or a mental state according to the measured data that is input, receives an evaluation for the estimated state of the subject, and performs learning processing of the machine learning model by using the evaluation for the estimated state of the subject.

### BACKGROUND

### 1. Technical Field

The present disclosure relates to an apparatus, a method, and a non-transitory computer-readable medium for performing learning processing of a machine learning model.

### 2. Related Art

Patent document 1 describes that “the information processing apparatus 100 can appropriately estimate information (for example, ideal biological information) related to an ideal physical condition of a user according to desired health information (for example, processed healthiness) indicating a health state that the user desires” (paragraph 0030).

Patent document 2 describes that “the estimation apparatus 40 estimates a facilitation type of resting brain waves, based on an output obtained by inputting the acquired resting-brain waves (EEG signals) in a trained model stored at step S21 (step S32)” (paragraph 0048).

### RELATED ART DOCUMENTS

### Patent Documents

Patent Document 1: Japanese Patent Application Publication No. 2023-180124

Patent Document 2: Japanese Patent Application Publication No. 2021-33524

### BRIEF DESCRIPTION OF THE DRAWINGS

FIG. 1 illustrates a configuration of a system 10 according to the present embodiment.

FIG. 2 illustrates an operational flow of the estimation apparatus 100 according to the present embodiment.

FIG. 3 illustrates an example of a screen 300 displayed on an output apparatus 30 according to the present embodiment.

FIG. 4 illustrates an example of a computer 1200 in which a plurality of aspects of the present invention may be embodied in whole or in part.

### DESCRIPTION OF EXEMPLARY EMBODIMENTS

The embodiments below do not limit the invention according to the claims. In addition, not all of the combinations of features described in the embodiments are essential to the solving means of the invention.

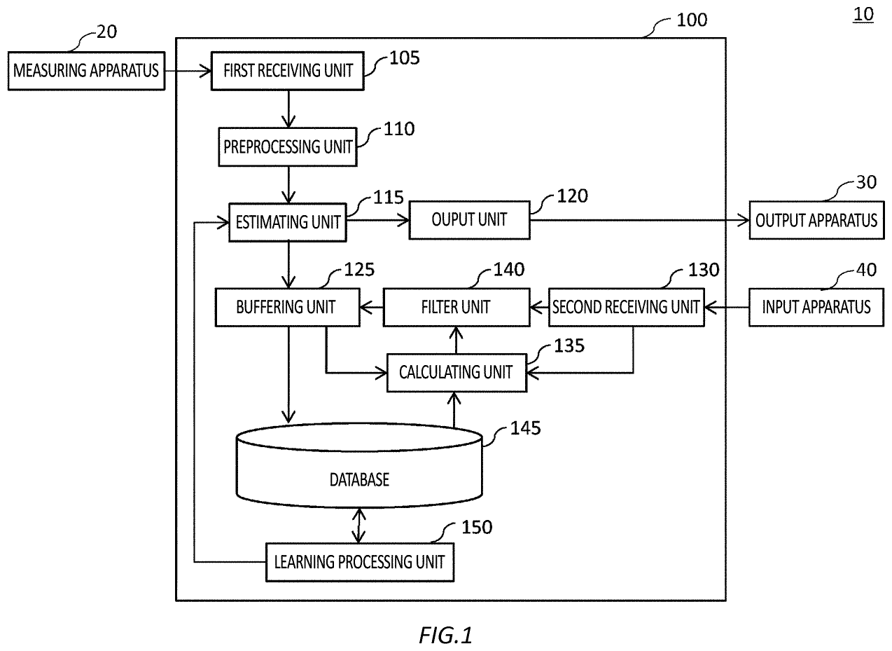

FIG. 1 illustrates a configuration of a system 10 according to the present embodiment. The system 10 includes a measuring apparatus 20, an output apparatus 30, an input apparatus 40, and an apparatus 100. The measuring apparatus 20 measures brain waves of a subject and supplies measured data of brain waves of the subject to the apparatus 100. The measuring apparatus 20 may be a wearable device. For example, the measuring apparatus 20 may be an ear-phone type, a necklace type, or a head-band type device. The measuring apparatus 20 may measure brain waves of the subject for each predetermined period.

The output apparatus 30 displays thereon a display screen output by the apparatus 100. The input apparatus 40 inputs an instruction for the apparatus 100 from a user of the system 10 or the like and supplies it to the apparatus 100. The output apparatus 30 and the input apparatus 40 may be implemented in a same computer, terminal apparatus, console apparatus, or the like, or may be implemented in a different computer, terminal apparatus, console apparatus or the like.

The apparatus 100 is connected to the measuring apparatus 20, the output apparatus 30, and the input apparatus 40. The apparatus 100 has a function of calculating an estimated state of the subject corresponding to the measured data of brain waves of the subject. Herein, the estimated state of the subject may be an estimated state of at least one of a physical state (fatigueness, physical condition, or the like) or a mental state (mental health, mood, physical condition, or the like). The apparatus 100 may be a computer such as a PC (personal computer), a tablet computer, a smartphone, a workstation, a server computer, or a general purpose computer, or may be a computer system in which a plurality of computers are connected. Such a computer system is also a computer in a broad sense. In addition, the apparatus 100 may be implemented by one or more virtual computer environments which can be run in the computer. Alternatively, the apparatus 100 may be a dedicated computer designed for the system 10, or may be a dedicated hardware achieved through a dedicated circuit. In the example of the present illustration, the apparatus 100 includes a first receiving unit 105, a preprocessing unit 110, a buffering unit 125, a second receiving unit 130, a calculating unit 135, a filter unit 140, and a database 145. Alternatively to the example of the present illustration, the apparatus 100 may not include some of the components.

The first receiving unit 105 is communicatively connected to the measuring apparatus 20. The first receiving unit 105 acquires the measured data of brain waves of the subject measured by the measuring apparatus 20 by means of wired communication or wireless communication. The first receiving unit 105 may acquire the measured data of brain waves of the subject from the measuring apparatus 20 by means of a communication circuitry or an input/output interface circuitry.

The preprocessing unit 110 is connected to the first receiving unit 105. The preprocessing unit 110 performs preprocessing of the measured data of brain waves of the subject acquired by the first receiving unit 105.

The estimating unit 115 is connected to the preprocessing unit 110. The estimating unit 115 estimates an estimated state of the subject according to the measured data of brain waves of the subject, in response to the measured data of brain waves of the subject after the preprocessing being input. The estimating unit 115 has a machine learning model which calculates, upon input of the measured data of brain waves, the estimated state of at least one of a physical state or a mental state according to the input measured data. The estimating unit 115 uses the machine learning model to input the measured data of brain waves of the subject to the machine learning model, and causes the estimated state according to the input measured data to be output from the machine learning model.

The output unit 120 is connected to the estimating unit 115. The output unit 120 outputs, to the output apparatus 30, the estimated state estimated by the estimating unit 115.

The buffering unit 125 is connected to the estimating unit 115. The buffering unit 125 buffers a time series of estimated states of the subject according to the measured data of brain waves of the subject. The buffering unit 125 may accumulate the estimated states of the subject according to the measured data of brain waves of the subject that is input from the estimating unit 115 every hour. The buffering unit 125 may erase, in response to the accumulated data being used, the estimated states of the subject according to the measured data of brain waves of the subject that is used.

The database 145 is connected to the buffering unit 125. The database 145 stores the measured data of brain waves of the subject, the estimated state of the subject according to said measured data, and an evaluation for the estimated state of the subject.

The second receiving unit 130 is communicatively connected to the input apparatus 40. The second receiving unit 130 acquires an evaluation for the estimated state of the subject input to the input apparatus 40 by means of wired communication or wireless communication. The second receiving unit 130 may acquire an evaluation by the user by receiving the evaluation by the user from the input apparatus 40 by means of the communication circuitry or the input/output interface circuitry. Herein, the “user” may be the subject, or may be another person.

The calculating unit 135 is connected to the buffering unit 125, the second receiving unit 130, and the database 145. The calculating unit 135 may acquire the evaluation for the estimated state of the subject from the second receiving unit 130. The calculating unit 135 may acquire, from the output unit 120, the measured data of brain waves of the subject corresponding to said estimated state. The calculating unit 135 may acquire, from the database 145, a measured data group including the measured data of brain waves of the subject and the estimated state of the subject, estimated for these pieces of measured data. The calculating unit 135 may calculate a distribution of a plurality of estimated states of the subject, estimated for the measured data group.

The filter unit 140 is connected to the buffering unit 125, the second receiving unit 130, and the calculating unit 135. The filter unit 140 may acquire an evaluation for the estimated state of the subject from the second receiving unit 130. The filter unit 140 may acquire the distribution of the plurality of estimated states of the subject from the calculating unit 135. The filter unit 140 may determine, based on the distribution of the plurality of estimated states, whether or not the evaluation for the estimated state of the subject acquired is an outlier. When the evaluation is an outlier, the filter unit 140 removes said evaluation.

The learning processing unit 150 is connected to the estimating unit 115 and the database 145. The learning processing unit 150 performs learning processing for the machine learning model of the estimating unit 115. The learning processing unit 150 may perform the learning processing for the machine learning model by using the measured data of brain waves of the subject stored in the database 145 and the evaluation for the estimated state of the subject according to said measured data, as the learning target.

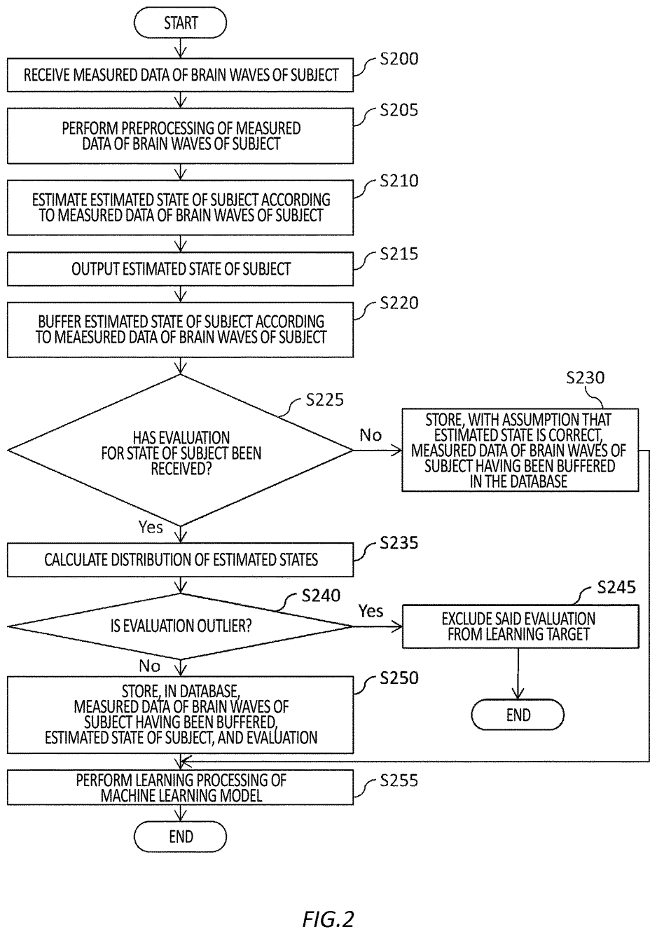

FIG. 2 illustrates an operational flow of the estimation apparatus 100 according to the present embodiment. At step 200 (S200), the first receiving unit 105 receives the measured data of brain waves of the subject. The first receiving unit 105 may acquire, as the measured data of brain waves of the subject, at least one of time series data of potentials measured from each of the electrodes provided at a plurality of locations on the head of said subject or time series data of a potential difference among the electrodes. The first receiving unit 105 may acquire at least one of time series data of a potential of each electrode or a spectrum obtained by converting the time series data of the potential difference among the electrodes into a frequency domain. The first receiving unit 105 may acquire an intensity for each predetermined frequency band extracted from potential difference data among electrodes. The first receiving unit 105 may acquire the measured data of brain waves of the subject from the measuring apparatus 20 and supply it to the preprocessing unit 110.

At S205, the preprocessing unit 110 performs preprocessing of the measured data of brain waves of the subject acquired by the first receiving unit 105. The preprocessing unit 110 may delete noises in the measured data. Herein, noises may originate from a movement of the subject during measurement. The preprocessing unit 110 may perform component decomposition by performing Fourier transform on the measured data. In this manner, the preprocessing unit 110 may calculate the intensity for each frequency band in the measured data. For example, the preprocessing unit 110 may calculate the intensity of at least one of an alpha wave, a beta wave, a gamma wave, a delta wave, or a theta wave included in the measured data of brain waves of the subject. When measured data of brain waves of the subject received by the first receiving unit 105 is already preprocessed, the apparatus 100 may not perform S205.

At S210, the estimating unit 115 estimates the estimated state of the subject according to the measured data of brain waves of the subject by using the machine learning model which calculates, upon input of the measured data of brain waves, the estimated state of at least one of a physical state or a mental state according to the input measured data. The estimating unit 115 may estimate the estimated state of the subject according to the measured data of brain waves, on which the preprocessing unit 110 performed preprocessing. Herein, the machine learning model may be any machine learning model to which a numeric value or the like is input and one or more values are output, or may be a neural network such as a convolution neural network (CNN), recurrent neural network (RNN), as examples, a regression model, or other machine learning models. Such a machine learning model can adjust internal parameters to bring the output data output according to the input data for learning closer to a target value, and thereby enable learning to output desired output data according to the input data. The machine learning model may be a model according to each subject, or may be a model according to all users of the system 10.

The neural network includes an input layer having a plurality of input nodes to receive an input value, one or more intermediate layers that receive information from the input layer to perform calculations, and an output layer having a plurality of output nodes to output a processed value. When the machine learning model used is a neural network, the estimating unit 115 may input the measured data of brain waves of the subject to each input node. For example, the estimating unit 115 may input the intensity for each frequency band (the intensity of the alpha wave, the beta wave, the gamma wave, the delta wave, or the theta wave) to each input node. Alternatively, the estimating unit 115 may input, to each input node, a spectrum intensity converted into a frequency domain. Alternatively, the estimating unit 115 may input a potential difference among electrodes during a certain time period. The machine learning model may have an output node corresponding to each of the one or more estimated states of the subject. The machine learning model may output, from each output node, a numeric value (0 to 1, 0 to 10 or the like) indicating a degree of each estimated state.

At S215, the output unit 120 outputs the estimated state of the subject estimated by the estimating unit 115. The output unit 120 may output only said estimated state (the latest estimated state) of the subject. Alternatively, the output unit 120 may output the estimated states of the subject in a time-series manner. For example, the first receiving unit 105 may receive measured data of brain waves of the subject at time t0, measured data of brain waves of the subject at time t1, and measured data of brain waves of the subject at time t2. The preprocessing unit 110 may perform preprocessing of these pieces of measured data. The estimating unit 115 may estimate the estimated state of the subject according to each of these pieces of measured data, that is, the estimated state of the subject at time t0, the estimated state of the subject at time t1, and the estimated state of the subject at time t2. The output unit 120 may output the estimated states of the subject in a time-series manner by outputting the estimated state of the subject at time t0, the estimated state of the subject at time t1, and the estimated state of the subject at time t2. When the output unit 120 outputs the estimated states of the subject in a time-series manner, the apparatus 100 can obtain an evaluation with higher accuracy compared to when only the latest estimated state is output.

At S220, the buffering unit 125 buffers a time series of the estimated states of the subject according to the measured data of brain waves of the subject. The buffering unit 125 may buffer the measured data of brain waves of the subject and the estimated states of the subject according to said measured data in association with each other. Alternatively to the example of the present illustration, the apparatus 100 may perform S220 before S215, or may perform S220 simultaneously with S215.

At S225, the second receiving unit 130 determines whether or not the evaluation for the estimated state of the subject has been received. The second receiving unit 130 may acquire the evaluation for the estimated state of the subject from the input apparatus 40. When the output unit 120 outputs the estimated states of the subject in a time-series manner, the second receiving unit 130 may receive the evaluation for relative temporal change in the estimated state of the subject. When the second receiving unit 130 receives the evaluation for the estimated state of the subject (Yes at S225), the apparatus 100 advances the processing to S235. Herein, the “evaluation” may be data indicating correctness or error of the estimated state, may be data indicating in what state the actual state of the subject is in compared to the estimated state (higher, lower, the same, or the like), or may be a numeric value indicating an actual state of the subject. The apparatus 100 may advance the processing to S235 only when the second receiving unit 130 receives the evaluation for the estimated state of the subject within a certain time period (such as within one hour) of the output thereof by the output unit 120. The second receiving unit 130 may consider the evaluation at one timing as the evaluation for the estimated state of the subject during a predetermined period up to the one timing.

At S235, the calculating unit 135 calculates a distribution of a plurality of estimated states of the subject, estimated for the measured data group including a plurality of pieces of measured data of brain waves of the subject. In response to the second receiving unit 130 receiving the evaluation for the estimated state of the subject, the calculating unit 135 may acquire, from the buffering unit 125, the measured data of brain waves of the subject corresponding to said estimated state. The calculating unit 135 may acquire, from the database 145, a data group including a plurality of pieces of measured data of brain waves of the subject acquired from the buffering unit 125 and a plurality of estimated states of the subject according to said plurality of pieces of measured data. The calculating unit 135 may calculate the distribution of the plurality of estimated states of the subject acquired from the database 145.

At S240, the filter unit 140 determines whether or not the evaluation for the estimated state of the subject received by the second receiving unit 130 is an outlier. The filter unit 140 determines whether or not the evaluation of the subject estimated according to the measured data included within a range of the measured data group is an outlier by using the distribution calculated by the calculating unit 135 at S235. In response to the evaluation for the estimated state of the subject not being included in the distribution calculated by the calculating unit 135, the filter unit 140 may determine that said evaluation is an outlier. In response to the evaluation for the estimated state of the subject being included in the distribution calculated by the calculating unit 135, the filter unit 140 may determine that said evaluation is not an outlier. In response to the deviation, in the evaluation for the estimated state of the subject, from the average value of the distribution calculated by the calculating unit 135 being equal to or greater than a predetermined threshold, the filter unit 140 may determine that said evaluation is an outlier. In response to the deviation, in the evaluation for the estimated state of the subject, from the average value of the distribution calculated by the calculating unit 135 being less than a predetermined threshold, the filter unit 140 may determine that said evaluation is not an outlier.

When the evaluation for the estimated state of the subject is an outlier (Yes at S240), the apparatus 100 advances the processing to S245. At S245, in response to the received evaluation being an outlier, the filter unit 140 excludes said evaluation from the learning target. In this manner, since data with low reliability is excluded from the learning target, the apparatus 100 can estimate the state of the subject more accurately. Alternatively to S245, the buffering unit 125 may store said evaluation in the database 145 as an outlier. In response to a predetermined number or more evaluations in which determination based on the same or similar representative data became an outlier being stored, the filter unit 140 may determine that these evaluations are not an outlier. Therefore, the filter unit 140 may include these evaluations in the learning target.

When the evaluation for the estimated state of the subject is not an outlier (No at S240), the apparatus 100 advances the processing to S250. The filter unit 140 may input, to the buffering unit 125, the evaluation for the estimated state of the subject.

At S250, the buffering unit 125 stores, in the database 145, the estimated state of the subject according to the measured data of brain waves of the subject buffered in the buffering unit 125 and the evaluation for said estimated state acquired from the filter unit 140. The buffering unit 125 may delete, from the buffering unit 125, the estimated state of the subject according to the measured data of brain waves of the subject stored in the database 145.

At S255, the learning processing unit 150 performs learning processing of the machine learning model by using the evaluation for the estimated state of the subject. The learning processing unit 150 may adjust learnable parameters in the machine learning model such that the estimated state of the subject output by the machine learning model in response to the measured data of brain waves of the subject being input becomes closer to the actual state of the subject indicated by the evaluation. When a neural network is used as the estimation model, the learning processing unit 150 may adjust a weight between each neuron of the neural network and a bias of each neuron or the like by means of back propagation or the like by using an error, relative to the actual state of the subject, of the estimated state of the subject output by the neural network in response to the measured data of brain waves of the subject being input. When the “evaluation” is data indicating correctness or error of the estimated state, the learning processing unit 150 may perform the learning processing of the machine learning model by using the estimated state for which an evaluation that “the estimated state is correct” has been performed and the corresponding measured data of brain waves. When the “evaluation” is data indicating what state the state of the subject is in compared to the estimated state, the learning processing unit 150 may calculate the actual state of the subject from the estimated state by using said evaluation. The learning processing unit 150 may perform the learning processing of the machine learning model by using the actual state of the subject calculated and the corresponding measured data of brain waves. When the “evaluation” is a numeric value indicating the actual state of the subject, the learning processing unit 150 may perform the learning processing of the machine learning model by using the actual state of the subject and the corresponding measured data of brain waves. The learning processing unit 150 may train the estimation model by using a known training algorithm for the machine learning model employed as the estimation model. The learning processing unit 150 may repeat the learning processing until an error such as a root mean squared percentage error (RMSPE) between the estimated state of the subject calculated by using at least a part of the training data and an actual state of the subject becomes equal to or lower than a predetermined threshold, or until training for a predetermined training hours or numbers of training is completed.

The learning processing unit 150 may acquire the data to be the learning target from the database 145. The learning processing unit 150 may update the machine learning model by performing further training on the trained machine learning model by using data to be the new learning target. Since data stored at S250 in may be included in the learning target acquired from the database 145, in response to the evaluation at one timing being received, the learning processing unit 150 may perform the learning processing of the machine learning model by using the evaluation and the measured data of brain waves of the subject buffered by the buffering unit 125 corresponding to the evaluation. The buffering unit 125 buffers the estimated state of the subject according to the measured data of brain waves of the subject, thereby the learning processing of the machine learning model can be performed by using the evaluation input after a certain time period has elapsed since the output by the output unit 120 is performed.

At S225, when the second receiving unit 130 considers the evaluation at one timing to be an evaluation for the estimated state of the subject during a predetermined period up to the one timing, in response to the evaluation at the one timing being received, the learning processing unit 150 may perform the learning processing of the machine learning model by using the evaluation for the one timing as the evaluation for the estimated state of the subject during the predetermined period up to the one timing. For example, in response to the evaluation at one timing being received, the buffering unit 125 may treat an evaluation at each timing that is being buffered to be the same as the evaluation at said one timing. In this manner, the learning processing unit 150 can perform the learning processing of the machine learning model by using more learning targets than the number of evaluations that are actually acquired. The learning processing unit 150 may set the learning rate in a case where the evaluation at the one timing is used as the evaluation of the estimated state of the subject during the predetermined period up to the one timing to be lower than the learning rate in a case where the evaluation at the one timing is used only as the evaluation at the one timing.

When the second receiving unit 130 does not receive the evaluation for the estimated state of the subject (No at S225), the apparatus 100 advances the processing to S230. At S230, the second receiving unit 130 may consider the evaluation that the estimated state of the subject is correct to have been acquired. This is because when the estimated state of the subject output by the output apparatus 30 is correct, there is a high possibility that the user will not actively input an evaluation to the input apparatus 40. In response to the second receiving unit 130 considering that an evaluation that the estimated state of the subject is correct has been acquired, the buffering unit 125 stores, with the assumption that the estimated state of the subject is correct, the estimated state of the subject according to the measured data of brain waves of the subject being buffered and the evaluation for said estimated state acquired from the second receiving unit 130 in the database 145.

At S255 after S230, the learning processing unit 150 performs the learning processing of the machine learning model by using the learning target acquired from the database 145. Since data stored at S230 is included in the learning target acquired from the database 145, in response to the evaluation for the estimated state of the subject not being received, the learning processing unit 150 will perform the learning processing of the machine learning model with the assumption that the estimated state of the subject is correct. In this manner, the apparatus 100 can perform the learning processing of the machine learning model by using data for a case where the estimated state of the subject is correct and the probability that the user will not actively input an evaluation is high.

The learning processing unit 150 may set the learning rate for a case where the learning processing of the machine learning model is performed under the assumption that the estimated state of the subject is correct to be lower than the learning rate for a case where the learning processing of the machine learning model is performed in response to an evaluation being received. In this manner, by setting the learning rate for a case where the evaluation may not be correct to be lower than the learning rate for a case where the evaluation is correct, the apparatus 100 can estimate the state of the subject more accurately.

According to the apparatus 100 shown above, in order to perform the learning processing of the machine learning model by using an evaluation for the estimated state of the subject, the estimated state of the subject can be estimated more accurately. According to the apparatus 100 shown above, the machine learning model for said subject can be optimized.

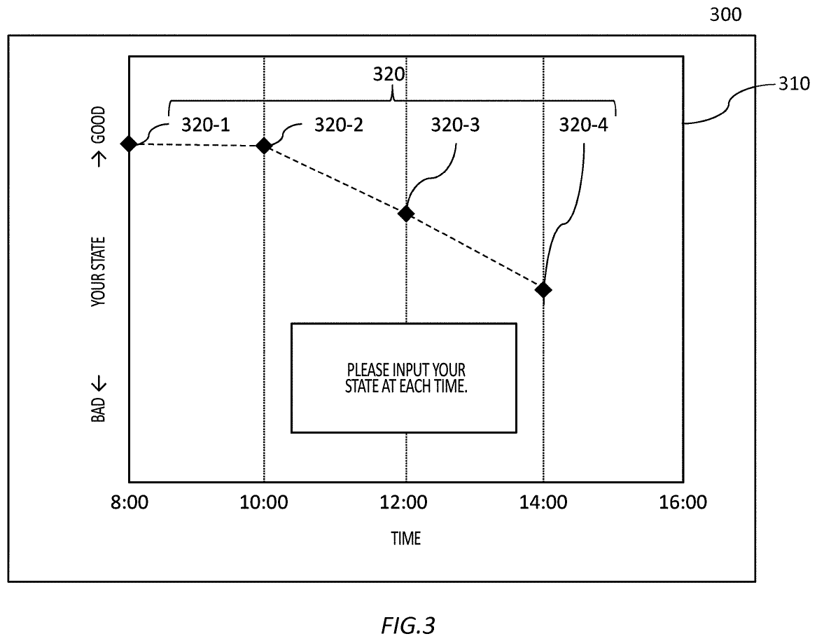

FIG. 3 illustrates an example of a screen 300 displayed on the output apparatus 30 according to the present embodiment. At S215 in FIG. 2, the output unit 120 outputs a screen 300 on the output apparatus 30. In the example of the present illustration, the screen 300 includes a graph 310. The graph 310 shows a time series of the estimated states of the subject. In the example of the present illustration, the horizontal axis of the graph 310 indicates the time, and the vertical axis thereof indicates the estimated state of the subject. The graph 310 has one or more points 320.

The point 320 indicates an estimated state of the subject at each time. In the example of the present illustration, the plot 320-1 indicates the estimated state of the subject at 8:00, the plot 320-2 indicates the estimated state of the subject at 10:00, the plot 320-3 indicates the estimated state of the subject at 12:00, and the plot 320-4 indicates the estimated state of the subject at 14:00. The point 320 may be a slider that is movable up and down in a vertical-axis direction in the graph 310. In response to the point 320 being moved up and down in the vertical-axis direction in the graph 310 and the movement being settled, the second receiving unit 130 may acquire the evaluation that the position of the point 320 after the movement indicates the actual state of the subject (S225 in FIG. 2).

Various embodiments of the present invention may be described with reference to flowcharts and block diagrams, where blocks may represent (1) stages of processes in which operations are executed or (2) sections of apparatuses responsible for executing operations. Certain stages and sections may be implemented by a dedicated circuit, a programmable circuit supplied together with computer-readable instructions stored on computer-readable media, and/or processors supplied together with computer-readable instructions stored on computer-readable media. The dedicated circuit may include digital and/or analog hardware circuits, and may include integrated circuits (IC) and/or discrete circuits. The programmable circuit may include a reconfigurable hardware circuit including logical AND, logical OR, logical XOR, logical NAND, logical NOR, and other logical operations, a memory element or the like such as a flip-flop, a register, a field programmable gate array (FPGA) and a programmable logic array (PLA), or the like.

A computer-readable medium may include any tangible device that can store instructions to be executed by a suitable device, and as a result, the computer-readable medium having instructions stored thereon includes a product including instructions that can be executed in order to create means for executing operations specified in the flowcharts or block diagrams. Examples of the computer-readable medium may include an electronic storage medium, a magnetic storage medium, an optical storage medium, an electromagnetic storage medium, a semiconductor storage medium, and the like. Examples of computer-readable media may include an electronic storage medium, a magnetic storage medium, an optical storage medium, an electromagnetic storage medium, a semiconductor storage medium, etc. More specific examples of computer-readable media may include a floppy disk (registered trademark), a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disk (DVD), a Blu-ray (registered trademark) disc, a memory stick, an integrated circuit card, etc.

The computer-readable instruction may include: an assembler instruction, an instruction-set-architecture (ISA) instruction; a machine instruction; a machine dependent instruction; a microcode; a firmware instruction; state-setting data; or either a source code or an object code described in any combination of one or more programming languages, including an object oriented programming language such as Smalltalk (registered trademark), JAVA (registered trademark), C++, or the like, and a conventional procedural programming language such as a “C” programming language or a similar programming language.

The computer-readable instruction may be provided for a processor or programmable circuit of a programmable data processing apparatus, such as a computer, locally or via a local area network (LAN), a wide area network (WAN) such as the Internet, or the like to execute the computer-readable instruction in order to create means for executing the operations specified in the flowcharts or block diagrams. Here, the computer may be a personal computer, or PC, a tablet computer, a smartphone, a workstation, a server computer, a general purpose computer, a special purpose computer, or the like, or may be a computer system to which a plurality of computers are connected. Such computer system to which the plurality of computers are connected is also referred to as a distributed computing system, and is a computer in a broad sense. In a distributed computing system, a plurality of computers collectively execute a program by each of the plurality of computers executing a portion of the program, and passing data during the execution of the program among the computers as needed.

Examples of the processor include a computer processor, a central processing unit (CPU), a processing unit, a microprocessor, a digital signal processor, a controller, a microcontroller, and the like. The computer may include one processor or a plurality of processors. In a multi-processor system including a plurality of processors, the plurality of processors collectively execute a program by each of the processors executing a portion of the program, and passing data during the execution of the program among the processors as needed. For example, in execution of multiple tasks, each of the plurality of processors may execute a portion of each task pieces by pieces by performing task-switching for each time slice. In this case, which portion of one program each processor is responsible for executing dynamically changes. Moreover, which portion of the program each of the plurality of processors is responsible for executing may be determined statically by multiprocessor-aware programming.

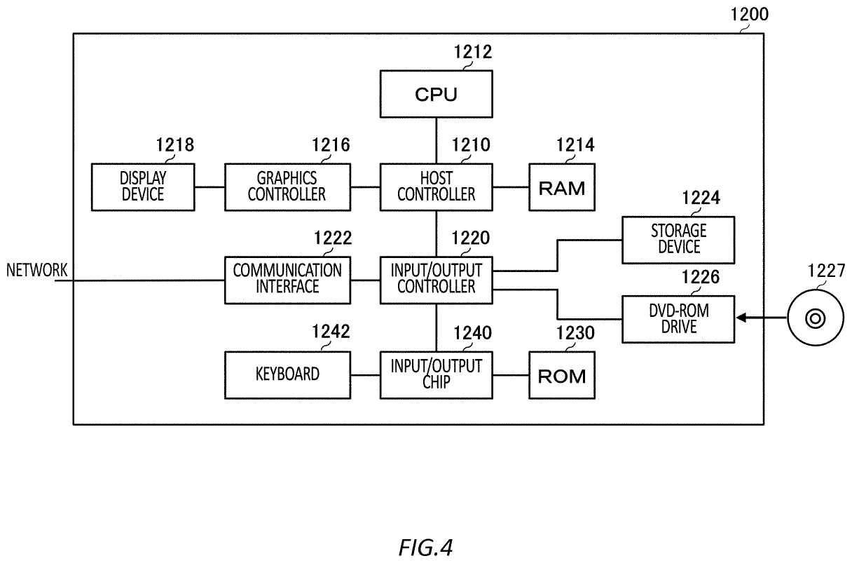

FIG. 4 illustrates an example of a computer 1200 in which a plurality of aspects of the present invention may be embodied in whole or in part. A program that is installed in the computer 1200 may cause the computer 1200 to function as operations associated with a device according to the embodiment of the present invention or one or more sections in the device, or may cause the computer 1200 to execute the operation or the one or more sections, and/or may cause the computer 1200 to execute processes according to the embodiment of the present invention or stages of the processes. Such a program may be executed by a CPU 1212 in order to cause the computer 1200 to execute particular operations associated with some or all of the blocks of flowcharts and block diagrams described herein.

The computer 1200 according to the present embodiment includes a CPU 1212, a RAM 1214, a graphics controller 1216, and a display device 1218, which are mutually connected by a host controller 1210. The computer 1200 also includes a communication interface 1222, a storage device 1224 such as a hard disk, input/output units such as a DVD-ROM drive 1226 and an IC card drive, which are connected to the host controller 1210 via an input/output controller 1220. The computer also includes legacy input/output units such as an ROM 1230 and a keyboard 1242, which are connected to the input/output controller 1220 via an input/output chip 1240.

The CPU 1212 operates according to programs stored in the ROM 1230 and the RAM 1214, thereby controlling each unit. The graphics controller 1216 acquires image data generated by the CPU 1212 on a frame buffer or the like provided in the RAM 1214 or in itself, and causes the image data to be displayed on a display device 1218.

The communication interface 1222 communicates with other electronic devices via a network. The storage device 1224 stores a program and data used by the CPU 1212 in the computer 1200. The DVD-ROM drive 1226 reads a program or data from a DVD-ROM 1227 and provides the program or data to the storage device 1224 via the RAM 1214. The IC card drive reads the programs and the data from the IC card, and/or writes the programs and the data to the IC card.

The ROM 1230 stores therein a boot program or the like that is executed by the computer 1200 at the time of activation, and/or a program which depends on the hardware of the computer 1200. The input/output chip 1240 may also connect various input/output units to the input/output controller 1220 via a parallel port, a serial port, a keyboard port, a mouse port, or the like.

Programs are provided by a computer-readable medium such as the DVD-ROM 1227 or the IC card. The programs are read from the computer-readable medium, are installed in the storage device 1224, the RAM 1214, or the ROM 1230, which are also an example of the computer-readable medium, and are executed by the CPU 1212. Information processing written in these programs is read by the computer 1200, and provides cooperation between the programs and the various types of hardware resources described above. A device or method may be constructed by realizing the operation or processing of information according to the use of the computer 1200.

For example, when communication is executed between the computer 1200 and an external device, the CPU 1212 may execute a communication program loaded onto the RAM 1214 to instruct communication processing to the communication interface 1222, based on the processing described in the communication program. Under the control of the CPU 1212, the communication interface 1222 reads transmission data stored in a transmission buffer processing region provided in a recording medium such as the RAM 1214, the storage device 1224, the DVD-ROM 1227, or the IC card, transmits the read transmission data to the network, or writes reception data received from the network in a reception buffer processing region or the like provided on the recording medium.

In addition, the CPU 1212 may cause the RAM 1214 to read all or a necessary portion of a file or database stored in an external recording medium such as the storage device 1224, the DVD-ROM drive 1226, or the DVD-ROM 1227, the IC card, or the like, and may execute various types of processes on data on the RAM 1214. The CPU 1212 may then write back the processed data to the external recording medium.

Various types of information such as various types of programs, data, tables, and databases may be stored in a recording medium and subjected to information processing. The CPU 1212 may execute various types of processing on the data read from the RAM 1214, which includes various types of operations, information processing, conditional judging, conditional branch, unconditional branch, search/replace of information, or the like, as described throughout this disclosure and designated by an instruction sequence of programs, and writes the result back to the RAM 1214. In addition, the CPU 1212 may search for information in a file, a database, or the like in the recording medium. For example, when a plurality of entries, each having an attribute value of a first attribute associated with an attribute value of a second attribute, are stored in the recording medium, the CPU 1212 may retrieve, out of the plurality of entries, an entry with the attribute value of the first attribute specified that meets a condition, read the attribute value of the second attribute stored in said entry, and thereby acquiring the attribute value of the second attribute associated with the first attribute satisfying a predetermined condition.

The above-described program or software module may be stored in the computer-readable medium on the computer 1200 or near the computer 1200. In addition, a recording medium such as a hard disk or a RAM provided in a server system connected to a dedicated communication network or the Internet may be used as the computer-readable medium, thereby providing the program to the computer 1200 via the network.

While the embodiments of the present invention have been described, the technical scope of the invention is not limited to the above-described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the operational flow is described by using phrases such as “first” or “next” in the claims, specification, or diagrams, it does not necessarily mean that the process must be performed in this order.

### Explanation of References

10: system, 20: measuring apparatus, 30: output apparatus, 40: input apparatus, 100: apparatus, 105: first receiving unit, 110: preprocessing unit, 115: estimating unit, 120: output unit, 125: buffering unit, 130: second receiving unit, 135: calculating unit, 140: filter unit, 145: database, 150: learning processing unit, 300: screen, 310: graph, 320: point, 1200: computer, 1210: host controller, 1212: CPU, 1214: RAM, 1216: graphics controller, 1218: display device, 1220: input/output controller, 1222: communication interface, 1224: storage device, 1226: DVD-ROM drive, 1227: DVD-ROM, 1230: ROM, 1240: input/output chip, 1242: keyboard.

What is claimed is:

1. An apparatus comprising a processor, wherein the processor:
receives measured data of brain waves of a subject;
estimates an estimated state of the subject according to the measured data of brain waves of the subject by using a machine learning model which calculates, upon input of measured data of brain waves, an estimated state of at least one of a physical state or a mental state according to the measured data that is input;
receives an evaluation for the estimated state of the subject; and
performs learning processing of the machine learning model by using the evaluation for the estimated state of the subject.

2. The apparatus according to claim 1, wherein the processor:
outputs the estimated state of the subject in a time-series manner; and
receives the evaluation for a relative temporal change in the estimated state of the subject in receiving the evaluation for the estimated state of the subject.

3. The apparatus according to claim 1, wherein in response to the evaluation received being an outlier, the processor excludes the evaluation from a learning target.

4. The apparatus according to claim 3 wherein the processor:
calculates a distribution of a plurality of estimated states of the subject estimated for measured data group including a plurality of pieces of measured data of brain waves of the subject; and
in excluding an evaluation from the learning target, determines, by using the distribution, whether or not an evaluation of the subject estimated according to the measured data included within a range of the measured data group is an outlier.

5. The apparatus according to claim 1, wherein the processor performs a learning processing of the machine learning model, in response to the evaluation at one timing being received in a learning processing of the machine learning model, by using the evaluation at the one timing as the evaluation for the estimated state of the subject during a predetermined period up to the one timing.

6. The apparatus according to claim 1, wherein the processor:
buffers a time series of the estimated states of the subject according to the measured data of brain waves of the subject; and
performs the learning processing of the machine learning model, in response to the evaluation at one timing being received in the learning processing of the machine learning model, by using the evaluation and the measured data of brain waves of the subject having been buffered, which corresponds to the evaluation.

7. The apparatus according to claim 1, wherein the processor performs the learning processing of the machine learning model, in response to the evaluation for the estimated state of the subject not being received in the learning processing of the machine learning model, assuming that the estimated state of the subject is correct.

8. The apparatus according to claim 7, wherein in learning processing of the machine learning model, the processor sets a learning rate in a case where learning processing of the machine learning model is performed under an assumption that the estimated state of the subject is correct to be lower than a learning rate in a case where learning processing of the machine learning model is performed in response to the evaluation being received.

9. A method comprising:
receiving, by a computer, measured data of brain waves of a subject;
estimating, by the computer, an estimated state of the subject according to the measured data of brain waves of the subject by using a machine learning model which calculates, upon input of measured data of brain waves, an estimated state of at least one of a physical state or a mental state according to the measured data that is input;
receiving, by the computer, an evaluation for the estimated state of the subject; and
performing, by the computer, learning processing of the machine learning model by using the evaluation for the estimated state of the subject.

10. The method according to claim 9, wherein the computer:
outputs the estimated state of the subject in a time-series manner; and
receives the evaluation for a relative temporal change in the estimated state of the subject in receiving the evaluation for the estimated state of the subject.

11. The method according to claim 9, wherein in response to the evaluation received being an outlier, the computer excludes the evaluation from a learning target.

12. The method according to claim 9, wherein the computer performs a learning processing of the machine learning model, in response to the evaluation at one timing being received in a learning processing of the machine learning model, by using the evaluation at the one timing as the evaluation for the estimated state of the subject during a predetermined period up to the one timing.

13. The method according to claim 9 wherein the computer:
buffers a time series of the estimated states of the subject according to the measured data of brain waves of the subject; and
performs the learning processing of the machine learning model, in response to the evaluation at one timing being received in the learning processing of the machine learning model, by using the evaluation and the measured data of brain waves of the subject having been buffered, which corresponds to the evaluation.

14. The method according to claim 9, wherein the computer performs the learning processing of the machine learning model, in response to the evaluation for the estimated state of the subject not being received in the learning processing of the machine learning model, assuming that the estimated state of the subject is correct.

15. A non-transitory computer-readable medium having recorded thereon a program that, when executed by a computer, causes the computer to execute processing to:
receive measured data of brain waves of a subject;
estimate an estimated state of the subject according to the measured data of brain waves of the subject by using a machine learning model which calculates, upon input of measured data of brain waves, an estimated state of at least one of a physical state or a mental state according to the measured data that is input;
receive an evaluation for the estimated state of the subject; and
perform learning processing of the machine learning model by using the evaluation for the estimated state of the subject.

16. The non-transitory computer-readable medium according to claim 15, wherein the computer:
outputs the estimated state of the subject in a time-series manner; and
receives the evaluation for a relative temporal change in the estimated state of the subject in receiving the evaluation for the estimated state of the subject.

17. The non-transitory computer-readable medium according to claim 15, wherein in response to the evaluation received being an outlier, the computer excludes the evaluation from a learning target.

18. The non-transitory computer-readable medium according to claim 15, wherein the computer performs a learning processing of the machine learning model, in response to the evaluation at one timing being received in a learning processing of the machine learning model, by using the evaluation at the one timing as the evaluation for the estimated state of the subject during a predetermined period up to the one timing.

19. The non-transitory computer-readable medium according to claim 15, wherein the computer:
buffers a time series of the estimated states of the subject according to the measured data of brain waves of the subject; and
performs the learning processing of the machine learning model, in response to the evaluation at one timing being received in the learning processing of the machine learning model, by using the evaluation and the measured data of brain waves of the subject having been buffered, which corresponds to the evaluation.

20. The non-transitory computer-readable medium according to claim 15, wherein the computer performs the learning processing of the machine learning model, in response to the evaluation for the estimated state of the subject not being received in the learning processing of the machine learning model, assuming that the estimated state of the subject is correct.
